(19) United States
(12) Patent Application Publication
(10) Pub. No.: US 2015/0200422 A1
(43) Pub. Date: Jul. 16, 2015

(54) ELECTROLYTE ADDITIVE FOR LITHIUM BATTERY, ELECTROLYTE INCLUDING THE SAME, AND LITHIUM BATTERY INCLUDING THE ELECTROLYTE

(71) Applicant: SAMSUNG SDI CO., LTD., Yongin-si (KR)

(72) Inventors: Jeong-Hye LEE, Yongin-si, Gyeonggi-do (KR); Woo-Cheol SHIN, Yongin-si, Gyeonggi-do (KR); Duck-Hyun KIM, Yongin-si, Gyeonggi-do (KR); Moon-Sung KIM, Yongin-si, Gyeonggi-do (KR); E-Rang CHO, Yongin-si, Gyeonggi-do (KR)

(21) Appl. No.: 14/535,817

(22) Filed: Nov. 7, 2014

(30) Foreign Application Priority Data
Jan. 16, 2014 (KR) 10-2014-0005605

(51) Int. Cl. H01M 10/0567; C07D 295/194; C07D 333/38; C07D 207/34; C07D 233/90; H01M 10/052; C07C 327/36

(52) U.S. Cl. CPC H01M 10/0567; H01M 10/052; C07D 295/194; C07C 327/36; C07D 207/34; C07D 233/90; C07D 333/38; H01M 2300/004

(57) ABSTRACT

The present invention relates to an electrolyte additive for a lithium battery, an electrolyte, and a lithium battery, the additive including a dithiocarbonyl-based compound represented by Formula 1:

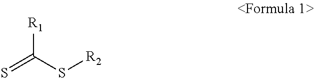

wherein, in Formula 1, R₁ is a substituted or unsubstituted alicyclic or aromatic hydrocarbon group, and R₂ is a substituted or unsubstituted aliphatic, alicyclic, or aromatic hydrocarbon group.

### CROSS-REFERENCE TO RELATED APPLICATION

Korean Patent Application No. 10-2014-0005605, filed on Jan. 16, 2014, in the Korean Intellectual Property Office, and entitled: “Electrolyte Additive For Lithium Battery, Electrolyte Including The Same, And Lithium Battery Using The Electrolyte,” is incorporated by reference herein in its entirety.

### BACKGROUND

1. Field

Embodiments relate to an electrolyte additive for a lithium battery, an electrolyte including the electrolyte additive, and a lithium battery including the electrolyte.

2. Description of the Related Art

With the advances in the field of small high-tech devices such as digital cameras, mobile devices, laptops, and personal computers, there has been a sharp demand increase for lithium batteries as energy sources. With the spread of electric cars, including hybrid electric vehicles (HEVs), plug-in hybrid electric vehicles (PHEVs), and electric vehicles (EVs), safe lithium batteries for these electric vehicles have been considered.

Lithium batteries may be operable at a high driving voltage because of a higher energy density per unit weight than that of lead storage batteries, nickel-cadmium (Ni—Cd) batteries, nickel-hydrogen batteries, and nickel-zinc batteries. Such lithium batteries may not be compatible with an aqueous electrolyte having high reactivity to lithium, and thus a nonaqueous organic electrolyte, along with a polar aprotic organic solvent for the nonaqueous organic electrolyte, may be used.

### SUMMARY

Embodiments are directed to an electrolyte additive for a lithium battery, an electrolyte including the electrolyte additive, and a lithium battery including the electrolyte.

The embodiments may be realized by providing an electrolyte additive for a lithium battery, the electrolyte additive comprising a dithiocarbonyl-based compound represented by Formula 1:

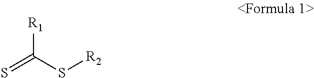

wherein, in Formula 1, R₁ includes a substituted or unsubstituted alicyclic or aromatic hydrocarbon group, and R₂ includes a substituted or unsubstituted aliphatic, alicyclic, or aromatic hydrocarbon group.

The aliphatic hydrocarbon group may be a C1-C20 alkyl group, a C2-C20 alkenyl group, or a C2-C20 alkynyl group, the alicyclic hydrocarbon group may be a C3-C20 cycloalkyl group, a C3-C20 cycloalkenyl group, or a C2-C20 heterocycloalkyl group, and the aromatic hydrocarbon group may be a C6-C20 aryl group or a C2-C20 heteroaryl group.

The C2-C20 heterocycloalkyl group and the C2-C20 heteroaryl group each independently comprise 1 to 3 heteroatoms.

The heteroatoms may include at least one of N, S, O, or P.

The C2-C20 heterocycloalkyl group may be a pyrolidinyl group, a piperidinyl group, a piperazinyl group, or a morpholinyl group, the C6-C20 aryl group may be a phenyl group or a naphthyl group, and the C2-C20 heteroaryl group may be a pyrrolyl group, an imidazolyl group, a pyrazolyl group, a benzimidazolyl group, a triazolyl group, a thiophenyl group, an indolyl group, a pyridinyl group, a pyridazinyl group, a pyrimidinyl group, or a quinolinyl group.

R₁ may be a pyrolidinyl group, a piperidinyl group, a piperazinyl group, a morpholinyl group, a phenyl group, a naphthyl group, a pyrrolyl group, an imidazolyl group, a pyrazolyl group, a benzimidazolyl group, a triazolyl group, or a thiophenyl group.

R₂ may be a methyl group, an ethyl group, a propyl group, an isopropyl group, a butyl group, an isobutyl group, a tert-butyl group, a sec-butyl group, a vinyl group, an allyl group, a butenyl group, a pentenyl group, a phenyl group, or a benzyl group.

The dithiocarbonyl-based compound represented by Formula 1 may include at least one of compound represented by Formula 2 to Formula 16:

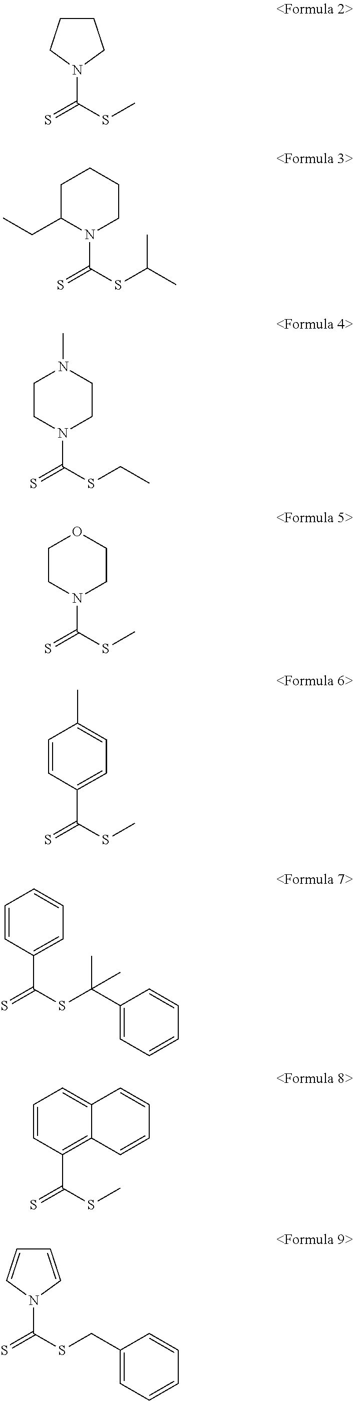

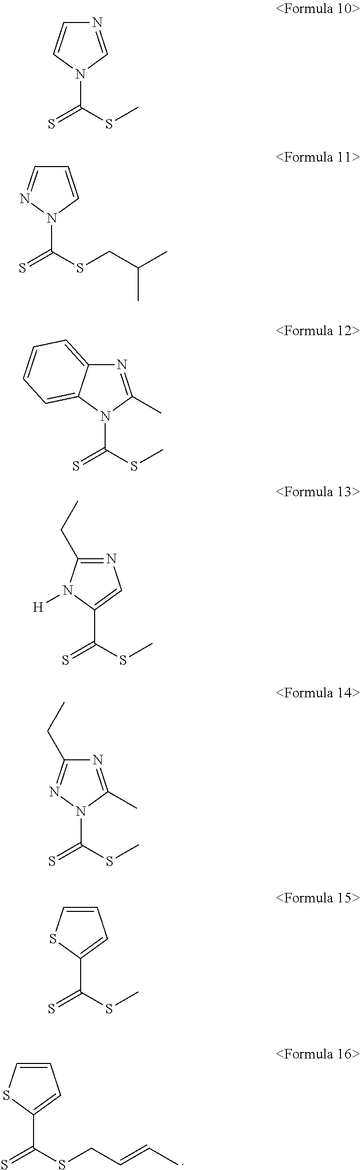

The embodiments may be realized by providing an electrolyte for a lithium battery, the electrolyte including a lithium salt; a nonaqueous organic solvent; and the electrolyte additive according to an embodiment.

The electrolyte additive may be included in the electrolyte in an amount of about 0.01 parts to about 5 parts by weight, based on 100 parts by weight of the nonaqueous organic solvent.

The electrolyte additive may be included in the electrolyte in an amount of about 0.1 parts to about 2 parts by weight, based on 100 parts by weight of the nonaqueous organic solvent.

The lithium salt may include at least one selected from the group of LiPF₆, LiBF₄, LiAsF₆, LiClO₄, LiCF₃SO₃, LiSbF₆, CF₃SO₃Li, LiN(SO₂CF₃)₂, LiC₄F₃SO₃, LiAlF₄, LiAlCl₄, LiN(SO₂C₂F₅)₂, LiN(C_{y}F₂ₓ₊₁SO₂)(C_{y}F_{2+y}SO₂), in which x and y are natural numbers, LiCl, and LiI.

The nonaqueous organic solvent may include a carbonate-based compound, an ester-based compound, an ether-based compound, a ketone-based compound, an alcohol-based compound, an aprotic compound, or a combination thereof.

The nonaqueous organic solvent may include the carbonate-based compound, the carbonate-based compound including a chain carbonate compound, a cyclic carbonate compound, a chain fluorocarbonate compound, a cyclic fluorocarbonate compound, or a combination thereof.

The nonaqueous organic solvent may include a chain carbonate compound and a cyclic carbonate compound.

The cyclic carbonate may be included in the electrolyte in an amount of at least about 5 vol. %, based on a total volume of the nonaqueous organic solvent.

The electrolyte may further include at least one additive selected from the group of tris(trimethylsilyl) phosphate (TMSPa), lithium difluorooxalate borate (LiFOB), vinylene carbonate (VC), fluoroethylene carbonate (FEC), vinyl ethylene carbonate (VEC), propane sultone (PS), succinonitrile (SN), LiBF₄, a silane compound having a functional group that forms a siloxane bond, and a silazane compound.

The electrolyte may further include propane sultone (PS), succinonitrile (SN), LiBF₄, or a combination thereof.

The embodiments may be realized by providing a lithium battery including a positive electrode; a negative electrode; and the electrolyte according to an embodiment.

### BRIEF DESCRIPTION OF THE DRAWINGS

Features will be apparent to those of skill in the art by describing in detail exemplary embodiments with reference to the attached drawings in which:

FIG. 1 illustrates a schematic view of a structure of a lithium battery according to an embodiment;

FIG. 2 illustrates a graph of differential capacity (dQ/dV) curves of lithium batteries of Example 5 and Comparative Example 1;

FIG. 3 illustrates a graph of impedances of the lithium batteries of Example 5 and Comparative Example 1; and

FIG. 4 illustrates a graph of temperature rate with respect to time in lithium batteries of Example 9 and Comparative example 6, obtained using an accelerating rate calorimeter (ARC).

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. Like reference numerals refer to like elements throughout.

As used herein, the term “and/or” includes any and all combinations of one or more of the associated listed items. Expressions such as “at least one of,” when preceding a list of elements, modify the entire list of elements and do not modify the individual elements of the list.

According to an embodiment, an electrolyte additive for a lithium battery may include a dithiocarbonyl-based compound represented by Formula 1 below.

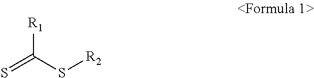

In Formula 1, R₁ may be a substituted or unsubstituted alicyclic or aromatic hydrocarbon group. R₂ may be a substituted or unsubstituted aliphatic, alicyclic, or aromatic hydrocarbon group.

An electrolyte of a lithium battery, e.g., a lithium secondary battery, may transport lithium ions from a positive electrode to a negative electrode during charging, and vice versa during discharging. When the electrolyte contacts the positive or negative electrode, oxidation-reduction decomposition may occur at an interface between the two, and the resulting decomposition product may be deposited or adsorbed onto a surface of the positive or negative electrode to form an interface layer. The decomposition product may be permanently adhered to serve as a protective layer of an electrode surface, though it may be partially desorbed or dissolved out of the interface layer. The protective layer may be substantially an insulator having negligible electron conductivity, but may have a high conductivity of lithium ions, and may behave like a solid electrolyte. For this reason, the protective layer may be called a “solid electrolyte interphase (SEI)” layer.

The SEI layer may serve as an ion tunnel in an electrode-electrolyte interface, and may relieve concentration deviations and an overvoltage to facilitate migration of lithium ions in a uniform current distribution. Once the SEI layer has been formed, the migration of electrons that induce a reaction between the electrolyte and at least one of the negative electrode and the positive electrode may be suppressed to thus prevent further decomposition of the electrolyte.

Thus, to help suppress an exothermic reaction in a lithium battery and to help increase a lifetime thereof, a stable SEI layer may be formed at the interface of the electrolyte. The additive according to an embodiment may help form an SEI layer having high-temperature stability and an electrolyte including the additive.

Hereinafter, an electrolyte additive for a lithium battery according to an embodiment will be described in greater detail for a better understanding.

The dithiocarbonyl-based compound represented by Formula 1 is an additive that is different from other additives used for electrolytes of other lithium (secondary) batteries (which may induce a decomposition reaction of an electrolyte on a surface of a positive electrode and a negative electrode to form an SEI layer that is not prone to deteriorate at a high temperature). For example, the dithiocarbonyl-based compound according to an embodiment may more easily accept electrons from a negative electrolyte, compared to polar organic solvents. For example, the dithiocarbonyl compound may be reduced at a voltage that is lower than a reduction voltage of a polar solvent, and thus may help suppress a reduction reaction of the electrolyte at an interface of the negative electrolyte. An SEI layer formed on a positive electrolyte may help effectively prevent permeation of an organic solvent into the positive electrode during intercalation of lithium ions to block direct contact between the organic solvent and the positive electrode, and consequentially may ensure safety of the lithium battery.

In the dithiocarbonyl-based compound represented by Formula 1 above, the aliphatic hydrocarbon group may include, e.g., a C1-C20 alkyl group, a C2-C20 alkenyl group, or a C2-C20 alkynyl group. The alicyclic hydrocarbon group may include, e.g., a C3-C20 cycloalkyl group, a C3-C20 cycloalkenyl group, or a C2-C20 heterocycloalkyl group. The aromatic hydrocarbon group may include, e.g., a C6-C20 aryl group or a C2-C20 heteroaryl group.

For example, the aliphatic hydrocarbon group may include a C1-C10 alkyl group, a C2-C10 alkenyl group, or a C2-C10 alkynyl group. For example, the alicyclic hydrocarbon group may include a C3-C10 cycloalkyl group, a C3-C10 cycloalkenyl group, or a C2-C10 heterocycloalkyl group. For example, the aromatic hydrocarbon group may include a C6-C10 aryl group or a C2-C10 heteroaryl group.

The heterocycloalkyl group and the heteroaryl group may each independently include one to three heteroatoms, e.g., which may include at least one selected from the group consisting N, S, O, P, and a combination thereof.

For example, the heterocycloalkyl group may include a pyrolidinyl group, a piperidinyl group, a piperazinyl group, or a morpholinyl group. For example, the aryl group may include a phenyl group or a naphthyl group. For example, the heteroaryl group may include a pyrrolyl group, an imidazolyl group, a pyrazolyl group, a benzimidazolyl group, a triazolyl group, a thiophenyl group, an indolyl group, a pyridinyl group, a pyridazinyl group, a pyrimidinyl group, or a quinolinyl group.

When R₁ in Formula 1 above is a alicyclic or aromatic hydrocarbon group, a cyclic form, rather than a chain form, may be directly linked to a dithiocarbonyl group, so that a resulting SEI layer may have improved stability even after a long charge and discharge. This may further prevent thermal decomposition of the electrolyte, and consequently help improve the safety of the lithium battery.

For example, R₁ may include a pyrolidinyl group, a piperidinyl group, a piperazinyl group, a morpholinyl group, a phenyl group, a naphthyl group, a pyrrolyl group, an imidazolyl group, a pyrazolyl group, a benzimidazolyl group, a triazolyl group, or a thiophenyl group.

For example, R₂ may include a methyl group, an ethyl group, a propyl group, an isopropyl group, a butyl group, an isobutyl group, a tert-butyl group, a sec-butyl group, a vinyl group, an allyl group, a butenyl group, a pentenyl group, a phenyl group, or a benzyl group.

In the dithiocarbonyl-based compound of Formula 1 above, the aliphatic, alicyclic, and aromatic hydrocarbon groups may each independently be substituted or unsubstituted. Substituents of the aliphatic, alicyclic, and aromatic hydrocarbon groups may each independently include a halogen atom, an amino group, a cyano group, a nitro group, a halogen-substituted or unsubstituted C1-C8 alkyl group; a halogen-substituted or unsubstituted C1-C8 alkoxy group; a halogen-substituted or unsubstituted C2-C8 alkenyl group; a halogen-substituted or unsubstituted C2-C8 alkenyloxy group; a halogen-substituted or unsubstituted C2-C8 alkynyl group; a halogen-substituted or unsubstituted C3-C10 cycloalkyl group; a halogen-substituted or unsubstituted C3-C10 cycloalkoxy group; a halogen-substituted or unsubstituted C3-C10 cycloalkenyl group; a halogen-substituted or unsubstituted C2-C10 heterocycloalkyl group; a halogen-substituted or unsubstituted C6-C10 aryl group; a halogen-substituted or unsubstituted C6-C10 aryloxy group; or a halogen-substituted or unsubstituted C6-C10 heteroaryl group. For example, substituents of the aliphatic, alicyclic, and aromatic hydrocarbon groups may each independently include at least one selected from the group of a halogen atom, a methyl group, an ethyl group, a propyl group, an isopropyl group, a butyl group, a tert-butyl group, a trifluoromethyl group, a tetrafluoroethyl group, a phenyl group, a naphthyl group, a tetrafluorophenyl group, a pyrrolyl group, and a pyridinyl group.

In an implementation, the dithiocarbonyl-based compound represented by Formula 1, above, may include at least one of compound represented by Formula 2 to Formula 16, below.

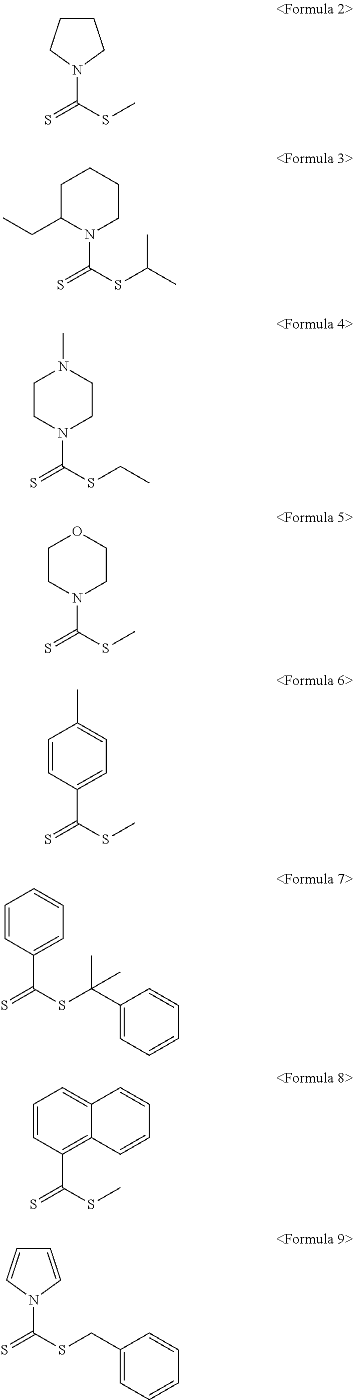

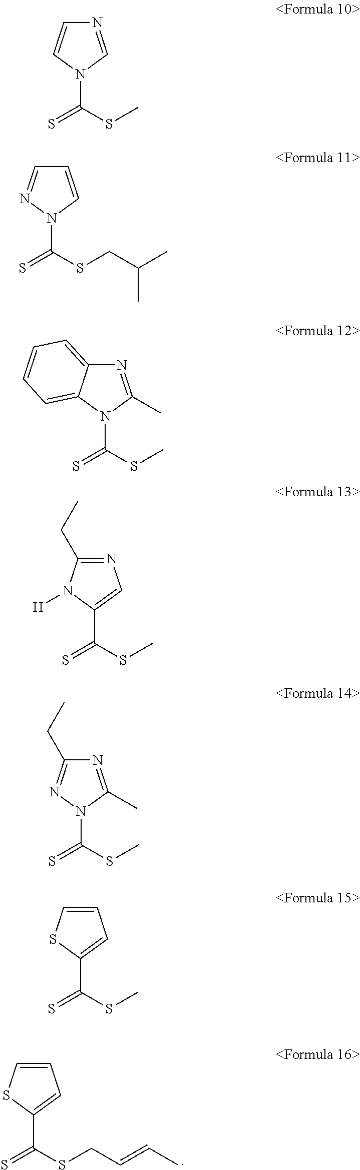

As used herein, the terminology of “a radical” may refer to a mono-radical or a di-radical. For example, a substituent for a group that may have two binding sites may be construed as a di-radical. For example, substituents for an alkyl group having two binding sites may include di-radicals, such as —CH₂₋, —CH₂CH₂₋, and —CH₂CH(CH₃)CH₂₋. The terminology of a radical such as “alkylene” may explicitly refer to a di-radical.

As used herein, the “alkyl group” or “alkylene group” may refer to a branched or unbranched aliphatic hydrocarbon group. In some embodiments, the alkyl group may be substituted or not. Examples of the alkyl group may include a methyl group, an ethyl group, a propyl group, an isopropyl group, a butyl group, an isobutyl group, a tert-butyl group, a pentyl group, a hexyl group, a cyclopropyl group, a cyclopentyl group, a cyclohexyl group, and a cycloheptyl group, which may be each independently substituted or not. In some embodiments, the alkyl group may have 1 to 10 carbon atoms. For example, the C1-C10 alkyl group may be a methyl group, an ethyl group, a propyl group, an isopropyl group, a butyl group, an iso-butyl group, a sec-butyl group, a pentyl group, a 3-pentyl group, a hexyl group, a heptyl group, an octyl group, a nonyl group, or a dodecyl group.

As used herein, the term “cycloalkyl group” may refer to a fully saturated carbocyclic or ring system, for example, a cyclopropyl group, a cyclobutyl group, a cyclopentyl group, or a cyclohexyl group.

As used herein, the term “alkenyl group” may refer to a hydrocarbon group including at least one carbon-carbon double bond. Examples of the alkenyl group may include an ethenyl group, a 1-propenyl group, a 2-propenyl group, a 2-methyl-1-propenyl group, a 1-butenyl group, and a 2-butenyl group. In some embodiments, the alkenyl group may be substituted or not. In some other embodiments, the alkenyl group may have 2 to 10 carbon atoms.

As used herein, the term “alkynyl group” may refer to a hydrocarbon group including at least one carbon-carbon triple bond. Examples of the alkynyl group may include an ethynyl group, a 1-propynyl group, a 1-butynyl group, and a 2-butynyl group. In some embodiments, the alkynyl group may be substituted or not. In some other embodiments, the alkynyl group may have 2 to 10 carbon atoms.

As used herein, the term “aromatic” may refer to a ring or a cyclic system having a conjugated π-electron system, for example, including a carbon cyclic aromatic ring and a heterocyclic aromatic ring. When the cyclic system is aromatic as a whole, the term “aromatic” may include a single ring or a fused polycyclic system (i.e., including a ring that shares adjacent electron pairs).

As used herein, the term “aryl group” may refer to an aromatic ring or cyclic system including only carbons in a backbone of the ring (i.e., at least two fused rings that share two adjacent carbon atoms). When an aryl group is a cyclic system, every ring in the cyclic system may be aromatic. Examples of the aryl group may include a phenyl group, a biphenyl group, a naphthyl group, a phenanthrenyl group, and a naphthacenyl group. The aryl group may be substituted or not.

As used herein, the term “heteroaryl group” may refer to an aromatic cyclic system having one ring or a plurality of fused rings in which at least one cyclic atom is a heteroatom, not carbon. In the cyclic system having fused rings, only one ring may include at least one heteroatom. Examples of the heteroatoms may include oxygen (O), sulfur (S), and nitrogen (N). Non-limiting examples of the heteroaryl group are a furanyl group, a thienyl group, an imidazolyl group, a quinazolinyl group, a quinolinyl group, an isoquinolinyl group, a quinoxalinyl group, a pyridinyl group, a pyrrolyl group, an oxazolyl group, and an indolyl group.

As used herein, the term “cycloalkenyl group” may refer to a carbocyclic ring or cyclic system having at least one double bond, but no aromatic ring. For example, the cycloalkenyl group may be a cyclohexenyl group.

As used herein, the term “heterocycloalkyl group” may refer to a nonaromatic ring or cyclic system including at least one heteroatom in a backbone of the ring.

As used herein, the term “halogen” may refer to a stable element of Group 17 of the periodic table of the elements, for example, fluorine, chlorine, bromine, or iodine. For example, the halogen may be fluorine and/or chlorine.

As used herein, a “substituent” may refer to a resulting product of exchange of at least one hydrogen of an unsubstituted, mother group with another atom or functional group. When a functional group is described as being “optionally substituted” herein, it means that the functional group may be substituted with a substituent as described above.

According to another embodiment, an electrolyte for a lithium battery may include a lithium salt, a nonaqueous organic solvent, and any of the electrolyte additives according to the above-described embodiments.

An amount of the dithiocarbonyl-based compound of Formula 1, above, (as an electrolyte additive) may be about 0.01 parts by weight to about 5 parts by weight, based on 100 parts by weight of the nonaqueous organic solvent. For example, a suitable amount of the dithiocarbonyl compound of Formula 1 above may be used as desired. In an implementation, the dithiocarbonyl-based compound of Formula 1 may be included in the electrolyte in an amount of about 0.1 parts by weight to about 5 parts by weight, e.g., about 0.1 parts by weight to about 3 parts by weight, about 0.1 parts by weight to about 2 parts by weight, or about 0.5 parts by weight to about 2 parts by weight, each based on 100 parts by weight of the nonaqueous organic solvent. When the amount of the dithiocarbonyl-based compound of Formula 1 is within these ranges, an SEI layer having an appropriate thickness and good safety may be obtained. Accordingly, a lithium battery with improved safety and without capacity reduction or resistance increase may be manufactured by using the electrolyte.

The lithium salt used in the electrolyte may serve as a supply source of lithium ions in the lithium battery, thereby enabling the basic operation of the lithium battery. The lithium salt may be any lithium salt suitably used in lithium batteries, e.g., a material dissolvable in any of the above-listed nonaqueous electrolytes. For example, the lithium salt may include LiCl, LiBr, LiI, LiClO₄, LiB₁₀Cl₁₀, LiPF₆, CF₃SO₃Li, CH₃SO₃Li, C₄F₃SO₃Li, (CF₃SO₂)₂NLi, LiN(CₓF₂ₓ₊₁SO₂)(C_{y}F_{2+y}SO₂) (in which x and y are natural numbers), CF₃CO₂Li, LiAsF₆, LiSbF₆, LiAlCl₄, LiAlF₄, lithium chloroborate, lower aliphatic lithium carbonate, 4-phenyl lithium borate, or lithium imide.

To help ensure a practical performance of a lithium battery, a concentration of the lithium salt in the electrolyte may be, e.g., about 0.1 M to about 2.0 M. When the concentration of the lithium salt is within this range, the electrolyte may have improved performance, a suitable conductivity and viscosity, and may help ensure effective migration of lithium ions.

The nonaqueous organic solvent used in the electrolyte may serve as a migration medium of ions involved in electrochemical reactions in the lithium battery. For example, the nonaqueous organic solvent may include a carbonate-based compound, an ester-based compound, an ether-based compound, a ketone-based compound, an alcohol-based compound, an aprotic solvent, or a combination thereof.

The carbonate-based compound may include a chain carbonate compound, a cyclic carbonate compound, a chain fluorocarbonate compound, a cyclic fluorocarbonate compound, or a combination thereof.

Examples of the chain carbonate compound may include diethyl carbonate (DEC), dimethyl carbonate, (DMC), dipropyl carbonate (DPC), methyl propyl carbonate (MPC), ethyl propyl carbonate (EPC), methyl ethyl carbonate (MEC), or a combination thereof.

Examples of the cyclic carbonate compound may include ethylene carbonate (EC), propylene carbonate (PC), butylene carbonate (BC), vinyl ethylene carbonate (VEC), or a combination thereof.

Examples of the chain or cyclic fluorocarbonate compound may include fluoroethylene carbonate (FEC), 4,5-difluoroethylene carbonate, 4,4-difluoroethylene carbonate, 4,4,5-trifluoroethylene carbonate, 4,4,5,5-tetrafluoroethylene carbonate, 4-fluoro-5-methylethylene carbonate, 4-fluoro-4-methylethylene carbonate, 4,5-difluoro-4-methylethylene carbonate, 4,4,5-trifluoro-5-methylethylene carbonate, trifluoromethylethylene carbonate, or a combination thereof.

The nonaqueous organic solvent may be a mixture of the chain carbonate compound and the cyclic carbonate compound. For example, when an amount of the cyclic carbonate compound is at least about 5 vol. %, based on a total volume of the nonaqueous organic solvent, cycle characteristics may be remarkably improved. In an implementation, the amount of the cyclic carbonate compound may be about 5vol. % to about 70vol. %, based on the total volume of the nonaqueous organic solvent. When the amount of the cyclic carbonate compound is within this range, the cyclic carbonate compound may have a specific dielectric constant of about 20 or greater to facilitate dissociation of the lithium salt, thereby further increasing the ion conductivity of the electrolyte.

The carbonate-based compound may include a mixture of the chain and/or cyclic carbonate compound and further include a fluorocarbonate compound. The fluorocarbonate compound may help increase the solubility of the lithium salt to help improve the ion conductivity of the electrolyte, and may facilitate formation of a film on the negative electrode. In an implementation, the fluorocarbonate compound may include FEC. An amount of the fluorocarbonate compound may be about 1 vol. % to about 30 vol. %, based on the total volume of the nonaqueous organic solvent. When the amount of the fluorocarbonate compound is within this range, a desired effect may be achieved due to a suitable viscosity of the nonaqueous organic solvent. In an implementation, the carbonate-based compound may further include VEC, together with FEC. For example, an amount of the VEC may be about 0.1 vol. % to about 10 vol. %, based on the total volume of the nonaqueous organic solvent.

Examples of the ester-based compound may include methyl acetate, ethyl acetate, n-propyl acetate, dimethyl acetate, methyl propionate (MP), ethyl propionate, γ-butyrolactone, decanolide, valerolactone, mevalonolactone, caprolactone, and methyl formate. Examples of the ether-based compound may include dibutyl ether, tetraglyme, diglyme, 1,2-dimethoxyethane, 1,2-diethoxyethane, ethoxymethoxyethane, 2-methyltetrahydrofuran, and tetrahydrofuran. An example of the ketone-based compound may include cyclohexanone. Examples of the alcohol-based compound may include ethyl alcohol and isopropyl alcohol.

Examples of the aprotic solvent may include dimethyl sulfoxide, 1,3-dioxolane, sulfolane, methyl sulfolane, 1,3-dimethyl-2-imidazolidinone, N-methyl-2-pyrrolidinone, formamide, dimethylformamide, acetonitrile, nitromethane, trimethyl phosphate, triethyl phosphate, trioctyl phosphate, and triester phosphate.

The above-listed nonaqueous organic solvents may be used alone or in a combination of at least two. In the latter, a mixing ratio of the at least two nonaqueous organic solvents may be appropriately adjusted depending on a desired performance of a battery.

In an implementation, the electrolyte may further include an additional additive, in addition to the dithiocarbonyl-based compound represented by Formula 1 above.

For example, the electrolyte may include vinylene carbonate (VC), catechol carbonate (CC), or the like, to form and maintain an SEI layer on the surface of the negative electrode.

In an implementation, the electrolyte may include a redox-shuttle additive, e.g., n-butyl ferrocene, a halogen-substituted benzene, or the like, to help prevent overcharging.

In an implementation, the electrolyte may include a film-forming additive, e.g., cyclohexyl benzene, biphenyl, or the like.

In an implementation, the electrolyte may include a cation receptor, e.g., a crown ether-based compound, and/or an anion receptor, e.g., a boron-based compound, to help improve conductive characteristics thereof.

In an implementation, the electrolyte may further include a phosphate-based compound as a flame retardant material, e.g., trimethyl phosphate (TMP), tris(2,2,2-trifluoroethyl) phosphate (TFP), hexamethoxy cyclotriphosphazene (HMTP), or the like.

The electrolyte may further include an additive to facilitate the formation of a stable SEI layer or film on the surface of an electrode to further improve the safety of the lithium battery, if desired. Examples of the additive may include tris(trimethylsilyl) phosphate (TMSPa), lithium difluorooxalato borate (LiFOB), propane sultone (PS), succinonitrile (SN), LiBF₄, a silane compound having a functional group able to form a siloxane bond (e.g., an acryl group, an amino group, an epoxy group, a methoxy group, an ethoxy group, or a vinyl group), and a silazane compound, such as hexamethy disilazane. For example, the additive may include PS, SN, LiBF₄, or the like.

The additives may be used alone or in a combination of at least two thereof.

For example, the above-listed cyclic carbonate compounds, such as VC, FEC, or VEC may be used as the electrolyte additive.

The other additives, e.g., other than the dithiocarbonyl-based compound of Formula 1 above, may be included in an amount of about 0.01 parts by weight to about 20 parts by weight, based on 100 parts by weight of the nonaqueous organic solvent. In an implementation, the other additives (other than the dithiocarbonyl-based compound of Formula 1 above), may be included in an amount of about 0.05 parts by weight to about 15 parts by weight, e.g., about 0.1 parts by weight to about 10 parts by weight or about 0.5 parts by weight to about 8 parts by weight, each based on 100 parts by weight of the nonaqueous organic solvent. In an implementation, the amount of the other additives may be sufficient to help ensure that the capacity retention rate of the lithium battery including the electrolyte is significantly reduced.

According to another embodiment, a lithium battery may include a positive electrode, a negative electrode, and an electrolyte according to an embodiment. For example, the lithium battery may include a positive electrode (including a positive active material), a negative electrode (opposite to the positive electrode and including a negative active material), a separator between the positive and negative electrodes, and the electrolyte according to the above-described embodiments.

A suitable lithium-containing metal oxide may be used as the positive active material. The positive active material may include at least one of composite oxides of lithium with a metal selected from among Co, Mn, Ni, and a combination thereof. For example, the positive electrode active material may include a compound represented by one of the following formulae: LiₐA_{1-b}B_{b}D₂ (where 0.90≦a≦1, and 0≦b≦0.5); LiₐE_{1-b}B_{b}O_{2-c}D_{c} (where 0.90≦a≦1, 0≦b≦0.5, and 0≦c≦0.05); LiE_{2-b}B_{b}O_{4-c}D_{c} (where 0≦b≦0.5, and 0≦c≦0.05); LiₐNi_{1-b-c}Co_{b}B_{c}D_{α} (where 0.90≦a≦1, 0≦b≦0.5, 0≦c≦0.05, and 0<α≦2); LiₐNi_{1-b-c}Co_{b}B_{c}O_{2-α}F_{α} (where 0.90≦a≦1, 0≦b≦0.5, 0≦c≦0.05, and 0<α<2); LiₐNi_{1-b-c}Co_{b}B_{c}O_{2-α}F₂ (where 0.90≦a≦1, 0≦b≦0.5, 0≦c≦0.05, and 0<a<2); LiₐNi_{1-b-c}Mn_{b}B_{c}D_{α} (where 0.90≦a≦1, 0≦b≦0.5, 0≦c≦0.05, and 0<α≦2); LiₐNi_{1-b-c}Mn_{b}B_{c}O_{2-α}F_{α} (where 0.90≦a≦1, 0≦b≦0.5, 0≦c≦0.05, and 0<α<2); LiₐNi_{1-b-c}Mn_{b}B_{c}O_{2-α}F₂ (where 0.90≦a≦1, 0≦b≦0.5, 0≦c≦0.05, and 0<α<2); LiₐNi_{b}E_{c}G_{d}O₂ (where 0.90≦a≦1, 0≦b≦0.9, 0≦c≦0.5, and 0.001≦d≦0.1); LiₐNi_{b}Co_{c}Mn_{d}GeO₂ (where 0.90≦a≦1, 0≦b≦0.9, 0≦c≦0.5, 0≦d≦0.5, and 0.001≦e≦0.1); LiₐNiG_{b}O₂ (where 0.90≦a≦1, and 0.001≦b≦0.1); LiₐCoG_{b}O₂ (where 0.90≦a≦1, and 0.001≦b≦0.1); LiₐMnG_{b}O₂ (where 0.90≦a≦1, and 0.001≦b≦0.1); LiₐMn₂G_{b}O₄ (where 0.90≦a≦1, and 0.001≦b≦0.1); QO₂; QS₂; LiQS₂; V₂O₅; LiV₂O₅; LiIO₂; LiNiVO₄; Li_{(3-f)}J₂(where 0≦f≦2); Li_{(3-f)}Fe₂(PO₄)₃ (where 0≦f≦2); and LiFePO₄.

In the formulae above, A may be selected from the group of nickel (Ni), cobalt (Co), manganese (Mn), and combinations thereof; B may be selected from the group of aluminum (Al), nickel (Ni), cobalt (Co), manganese (Mn), chromium (Cr), iron (Fe), magnesium (Mg), strontium (Sr), vanadium (V), a rare earth element, and combinations thereof; D may be selected from the group of oxygen (O), fluorine (F), sulfur (S), phosphorus (P), and combinations thereof; E may be selected from the group of cobalt (Co), manganese (Mn), and combinations thereof; F may be selected from the group of fluorine (F), sulfur (S), phosphorus (P), and combinations thereof; G may be selected from the group of aluminum (Al), chromium (Cr), manganese (Mn), iron (Fe), magnesium (Mg), lanthanum (La), cerium (Ce), strontium (Sr), vanadium (V), and combinations thereof; Q may be selected from the group of titanium (Ti), molybdenum (Mo), manganese (Mn), and combinations thereof; I may be selected from the group of chromium (Cr), vanadium (V), iron (Fe), scandium (Sc), yttrium (Y), and combinations thereof; and J may be selected from the group of vanadium (V), chromium (Cr), manganese (Mn), cobalt (Co), nickel (Ni), copper (Cu), and combinations thereof.

For example, the positive electrode active material may include LiCoO₂, LiMnₓO₂ₓ (where x=1 or 2), LiNi₁₋ₓMnₓO₂ (where 0<x<1), LiNi_{1-x-y}CoₓMn_{y}O₂ (where 0≦x≦0.5, 0≦y≦0.5), or FePO₄.

In an implementation, the compounds listed above as positive electrode active materials may have a surface coating layer (hereinafter, “coating layer”). In an implementation, a mixture of a compound without a coating layer and a compound having a coating layer, the compounds being selected from the compounds listed above, may be used. The coating layer may include at least one compound of a coating element selected from the group of oxide, hydroxide, oxyhydroxide, oxycarbonate, and hydroxycarbonate of the coating element. The compounds for the coating layer may be amorphous or crystalline. The coating element for the coating layer may include magnesium (Mg), aluminum (Al), cobalt (Co), potassium (K), sodium (Na), calcium (Ca), silicon (Si), titanium (Ti), vanadium (V), tin (Sn), germanium (Ge), gallium (Ga), boron (B), arsenic (As), zirconium (Zr), or mixtures thereof. The coating layer may be formed using a suitable that does not adversely affect the physical properties of the positive electrode active material when a compound of the coating element is used. For example, the coating layer may be formed using a spray coating method, a dipping method, or the like.

For example, the positive electrode may be manufactured by dispersing such a positive active material as described above, a binder, and optionally a conducting agent in a solvent to prepare a positive active material layer composition, and then molding the positive active material layer composition into a certain shape, or coating the positive active material layer composition on a current collector such as an aluminum foil.

The binder in the positive active material layer composition may facilitate binding between the positive active material and the conducting agent, and binding of the positive active material to the current collector. The binder may be may be included in an amount of about 1 to about 50 parts by weight, based on 100 parts by weight of the total weight of the positive active material. In an implementation, the amount of the binder may be about 1 part to about 30 parts by weight, e.g., about 1 part to about 20 parts by weight or about 1 part to about 15 parts by weight, each based on 100 parts by weight of the positive active material. Examples of the binder may include polyvinylidene fluoride (PVDF), polyvinylidene chloride, polybenzimidazole, polyimide, polyvinyl acetate, polyacrylonitrile, polyvinyl alcohol, carboxymethyl cellulose (CMC), starch, hydroxypropyl cellulose, regenerated cellulose, polyvinylpyrrolidone, tetrafluoroethylene, polyethylene, polypropylene, polystyrene, polymethyl methacrylate, polyaniline, acrylonitrile butadiene styrene, phenol resin, epoxy resin, polyethylene terephthalate, polytetrafluoroethylene, polyphenylene sulfide, polyamide imide, polyether imide, polyethylene sulfone, polyamide, polyacetal, polyphenylene oxide, polybutylene terephthalate, ethylene-propylene-diene monomer (EPDM), sulfonated EPDM, SBR, fluoro rubber, or a combination thereof.

In an implementation, the positive electrode may further include a conducting agent that provides a conduction path to the positive active material to further improve electrical conductivity. The conducting agent may include one suitably used in lithium batteries. Examples of the conducting agent may include carbonaceous materials, such as natural graphite, artificial graphite, carbon black, acetylene black, ketchen black, carbon fibers, and the like; metal-based materials, such as copper, nickel, aluminum, silver, and the like, in powder or fiber form; and conductive materials, including conductive polymers, such as a polyphenylene derivative; or a mixture thereof. The amount of the conducting agent may be appropriately adjusted. For example, a weight ratio of the positive active material to the conducting agent may be about 99:1 to about 90:10.

Examples of the solvent may include N-methylpyrrolidone (NMP), acetone, and water. An appropriate amount of the solvent may be used to facilitate coating of the current collector.

The current collector may have a thickness of about 3 μm to about 500 μm. The current collector may include suitable materials that provide a suitable conductivity without causing chemical changes in a fabricated battery. Examples of materials for the current collector may include copper, stainless steel, aluminum, nickel, titanium, sintered carbon, copper or stainless steel that is surface-treated with carbon, nickel, titanium, or silver, and aluminum-cadmium alloys. In an implementation, the current collector for the positive electrode may be processed to have fine irregularities on surfaces thereof so as to enhance the adhesive strength of the current collector to the positive active material, and may be used in any of various forms including films, sheets, foils, nets, porous structures, foams, and non-woven fabrics.

The positive electrode may be manufactured by directly coating the positive active material layer composition on an aluminum current collector to form a positive electrode plate and drying and pressing the positive electrode plate. Alternatively, the positive electrode may be manufactured by casting the positive active material layer composition on a separate support to form a positive active material film, separating the positive active material film from the support, laminating the positive active material film on an aluminum current collector to form a positive electrode plate, and drying and pressing the positive electrode plate.

The negative electrode may be manufactured as follows: The negative electrode may be manufactured in the same manner as that of the positive electrode, except for using a negative active material, instead of the positive active material. A binder, a conducting agent, and a solvent for a negative active material layer composition may be the same as those used in the positive active material layer composition.

In an implementation, the negative electrode may be manufactured by directly coating the negative active material layer composition on a copper current collector to form a negative electrode plate, and drying and pressing the negative electrode plate. In an implementation, the negative electrode may be manufactured by casting the negative active material layer composition on a separate support to form a negative active material film, separating the negative active material film from the support, laminating the negative active material film on a copper current collector to form a negative electrode plate, and drying and pressing the negative electrode plate.

The negative active material may be a suitable negative active material for a lithium battery. For example, the negative active material may include at least one selected from the group of lithium metal, a metal that is alloyable with lithium, a transition metal oxide, a non-transition metal oxide, and a carbonaceous material.

Examples of the metal alloyable with lithium may include Si, Sn, Al, Ge, Pb, Bi, Sb, a Si—Y alloy (where Y is an alkali metal, an alkali earth metal, a Group XIII element, a Group XIV element, a transition metal, a rare earth element, or a combination thereof except for Si), and a Sn—Y alloy (where Y is an alkali metal, an alkali earth metal, a Group XIII element, a Group XIV element, a transition metal, a rare earth element, or a combination thereof except for Sn). Y may be magnesium (Mg), calcium (Ca), strontium (Sr), barium (Ba), radium (Ra), scandium (Sc), yttrium (Y), titanium (Ti), zirconium (Zr), hafnium (Hf), rutherfordium (Rf), vanadium (V), niobium (Nb), tantalum (Ta), dubnium (Db), chromium (Cr), molybdenum (Mo), tungsten (W), seaborgium (Sg), technetium (Tc), rhenium (Re), bohrium (Bh), iron (Fe), lead (Pb), ruthenium (Ru), osmium (Os), hassium (Hs), rhodium (Rh), iridium (Ir), palladium (Pd), platinum (Pt), copper (Cu), silver (Ag), gold (Au), zinc (Zn), cadmium (Cd), boron (B), aluminum (Al), gallium (Ga), tin (Sn), indium (In), titanium (Ti), germanium (Ge), phosphorus (P), arsenic (As), antimony (Sb), bismuth (Bi), sulfur (S), selenium (Se), tellurium (Te), polonium (Po), or combinations thereof.

Examples of the transition metal oxide may include a lithium titanium oxide, a vanadium oxide, and a lithium vanadium oxide.

Non-limiting examples of the non-transition metal oxide are SnO₂ and SiOₓ (where 0<x<2).

Examples of the carbonaceous material are crystalline carbon, amorphous carbon, or mixtures thereof. Examples of the crystalline carbon may include graphite, such as natural graphite or artificial graphite that is in amorphous, plate, flake, spherical, or fibrous form. Examples of the amorphous carbon may include soft carbon, hard carbon, meso-phase pitch carbides, sintered corks, and the like.

Next, a separator (to be disposed between the positive electrode and the negative electrode) may be prepared. The positive electrode and the negative electrode may be separated from each other by the separator. A suitable separator for lithium batteries may be used. In an implementation, the separator may have low resistance to migration of ions in the electrolyte and a high electrolyte-retaining ability. Examples of materials for the separator may include glass fiber, polyester, Teflon, polyethylene, polypropylene, polytetrafluoroethylene (PTFE), and a combination thereof, each of which may be a nonwoven fabric or a woven fabric. The separator may have a pore diameter of about 0.01 μm to about 10 μm and a thickness of about 5 μm to about 300 μm.

After forming an electrode assembly having a layer-built cell structure in which bicell structures having structure of a positive electrode/a separator/a negative electrode/a separator/a positive electrode, or unit cell structures are repeatedly disposed upon one another, the electrode assembly may be encased in a cylindrical case, the electrolyte may be injected thereinto and a cap may be used to seal a lithium battery, and thus complete the manufacture of the lithium battery described above.

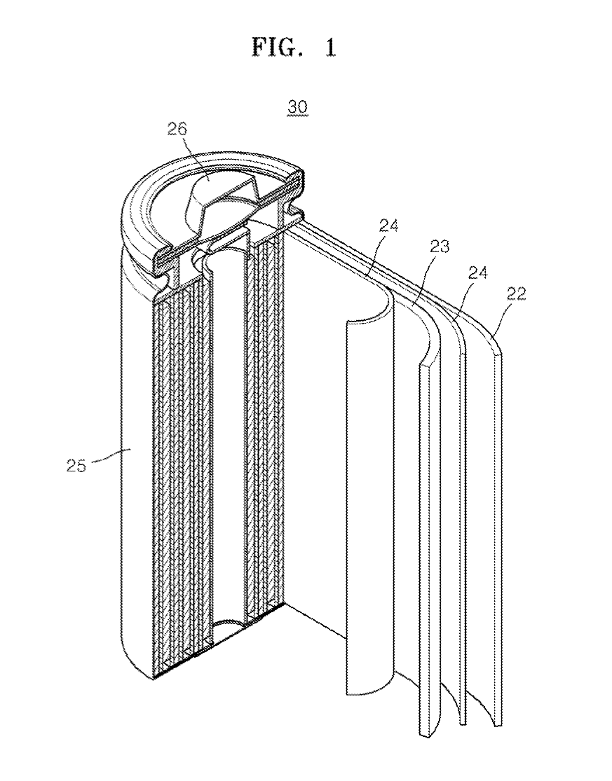

A lithium battery 30 according to an embodiment is illustrated in FIG. 1. Referring to FIG. 1, the lithium battery 30 may include a positive electrode 23, a negative electrode 22, and a separator 24 between the positive electrode 23 and the negative electrode 22. The positive electrode 23, the negative electrode 22, and the separator 24 may be wound or folded, and then accommodated in a battery case 25. Subsequently, an electrolyte may be injected into the battery case 25, and the battery case 25 may be sealed with a cap assembly member 26 and thus may complete the manufacture of the lithium battery 30. The battery case 25 may have a cylindrical shape, a rectangular shape, or a thin-film shape. The lithium battery 30 may be a lithium ion battery.

The lithium batteries may be classified as, e.g., a winding type or a stack type, depending on a structure of electrodes, or as, e.g., a cylindrical type, a rectangular type, a coin type, or a pouch type, depending on a type of exterior shape thereof.

Lithium batteries may be used as power sources of small devices, and/or as unit cells of medium- or large-sized battery devices, each module consisting of a plurality of cells.

Examples of the medium- or large-sized devices may include power tools; electric cars, including electric vehicles (EVs), hybrid electric vehicles (HEVs), plug-in hybrid electric vehicles (PHEVs); electric two-wheeled vehicles, including E-bikes and E-scooters; electric golf carts; electric trucks; electric commercial vehicles, and power storage systems. In an implementation, the lithium battery may be used in applications that require high-power output, high voltage, and operate under high-temperature conditions.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

(Preparation of Electrolyte)

### Preparation Example 1

After LiPF₆ was dissolved to a concentration of 1.3 M in a mixed nonaqueous organic solvent of EC, ethylmethyl carbonate (EMC), and dimethyl carbonate (DMC) in a volume ratio of 10:10:80, 5 parts by weight of fluoroethyl carbonate (FEC), 0.5 parts by weight of VEC, 1 part by weight of SN, 0.2 parts by weight of LiBF₄, and 1 part by weight of 1-pyrrolidinecarbodithioic acid (represented by Formula 2 below) were added as additives thereto to prepare an electrolyte.

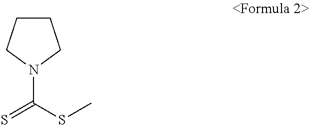

### Preparation Example 2

An electrolyte was prepared in substantially the same manner as in Preparation Example 1, except that 1 part by weight of methyl 4-morpholinecarbodithioate (represented by Formula 5 below) was added as an additive instead of the compound of Formula 2.

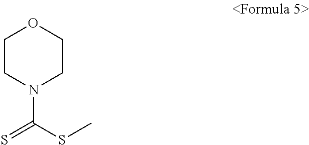

### Preparation Example 3

An electrolyte was prepared in substantially the same manner as in Preparation Example 1, except that 1 part by weight of naphthalene-1-carbodithioic acid methyl ester (represented by Formula 8 below) was added as an additive instead of the compound of Formula 2.

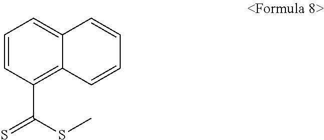

### Preparation Example 4

An electrolyte was prepared in substantially the same manner as in Preparation Example 1, except that 1 part by weight of phenylmethyl pyrrole-1-carbodithioate (represented by Formula 9 below) was added as an additive instead of the compound of Formula 2.

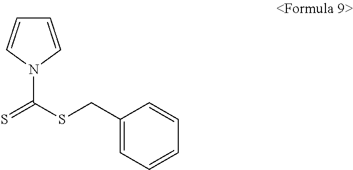 [SMILES: S=C(SCc1ccccc1)n1cccc1]

### Preparation Example 5

An electrolyte was prepared in substantially the same manner as in Preparation Example 1, except that 1 part by weight of 1-(methyldithiocarbonyl) imidazole (represented by Formula 10 below) was added as an additive instead of the compound of Formula 2.

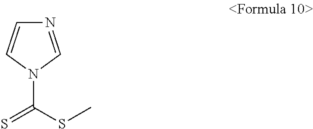

### Preparation Example 6

An electrolyte was prepared in substantially the same manner as in Preparation Example 1, except that 0.5 parts by weight of 1-(methyldithiocarbonyl) imidazole (represented by Formula 10 above) was added as an additive instead of the compound of Formula 2.

### Preparation Example 7

An electrolyte was prepared in substantially the same manner as in Preparation Example 1, except that 2 parts by weight of 1-(methyldithiocarbonyl) imidazole (represented by Formula 10 above) was added as an additive instead of the compound of Formula 2.

### Preparation Example 8

An electrolyte was prepared in substantially the same manner as in Preparation Example 1, except that 1 part by weight of methyl 2-thiophenecarbodithioate (represented by Formula 15 below) was added as an additive instead of the compound of Formula 2.

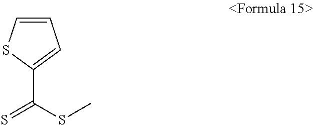

### Comparative Preparation Example 1

An electrolyte was prepared in substantially the same manner as in Preparation Example 1, except that 1-pyrrolidinecarbodithioic acid (represented by Formula 2 above) was not added.

### Comparative Preparation Example 2

An electrolyte was prepared in substantially the same manner as in Preparation Example 1, except that 1 part by weight of methyl 2-methylpropanedithioate (represented by Formula 17 below) was added as an additive, instead of the compound of Formula 2.

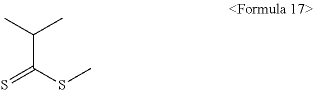

### Comparative Preparation Example 3

An electrolyte was prepared in substantially the same manner as in Preparation Example 1, except that 1 part by weight of methyl 2-methoxyethanedithioate (represented by Formula 18 below) was added as an additive instead of the compound of Formula 2.

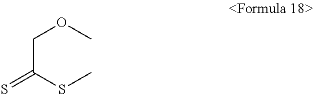

### Comparative Preparation Example 4

An electrolyte was prepared in substantially the same manner as in Preparation Example 1, except that 1 part by weight of methyl diethyldithiocarbamate (represented by Formula 19 below) was added as an additive instead of the compound of Formula 2.

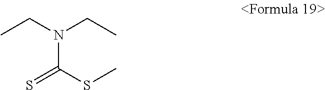

Comparative Preparation Example 5

An electrolyte was prepared in substantially the same manner as in Preparation Example 1, except that 1 part by weight of O-benzylhydryl S-methyl xanthate (represented by Formula 20 below) was added as an additive instead of the compound of Formula 2.

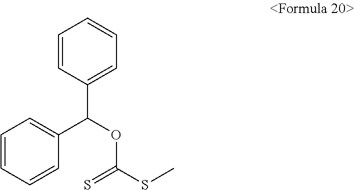 [SMILES: CSC(=S)OC(c1ccccc1)c1ccccc1]

(Manufacture of Test Cells)

### Example 1

(Manufacture of Positive Electrode)

LiNi_{0.5}Co_{0.3}Mn_{0.2}O₂ as a positive active material, PVDF as a binder, and denka black as a conducting agent were mixed in a weight ratio of 92:4:4, and then dispersed in N-methyl-2-pyrrolidone to prepare a positive active material layer composition. The positive active material layer composition was coated on an aluminum foil current collector having a thickness of about 20 μm, dried and then pressed to manufacture a positive electrode.

(Manufacture of Negative Electrode)

Graphite as a negative active material, and SBR and CMC as binders were mixed in a weight ratio of 97.5:1.5:1.0, and then dispersed in N-methyl-2-pyrrolidone to prepare a negative active material layer composition. The negative active material layer composition was coated on a copper foil current collector having a thickness of about 15 μm, dried and then pressed to manufacture a negative electrode.

(Manufacture of Lithium Secondary Battery)

The positive electrode, the negative electrode, a double-layered polyethylene (PE) separator having a thickness of about 18 μm, and the electrolyte of Preparation Example 1 were used to manufacture a 18650 standard cylindrical battery.

### Examples 2 to 8

Positive electrodes, negative electrodes, and lithium secondary batteries were manufactured in substantially the same manner as in Example 1, except that the electrolytes of Preparation Examples 2 to 8 were used, respectively.

### Comparative Examples 1 to 5

Positive electrodes, negative electrodes, and lithium secondary batteries were manufactured in substantially the same manner as in Example 1, except that the electrolytes of Comparative Preparation Examples 1 to 5 were used, respectively.

### Evaluation Example 1

### Battery Safety Evaluation—Penetration Test

A penetration test was performed on the lithium secondary batteries of Examples 1 to 8 and Comparative Examples 1 to 5 as described below. The results are shown in Table 1, below.

After the lithium secondary batteries were subjected to charging under a standard condition (at 0.5 C to 4.2 V, and 0.05 C (cut-off)) and then left to rest for about 10 minutes or longer, each of the lithium secondary batteries was completely penetrated through the middle thereof with a nail (a 2.5-mm diameter) at a rate of about 80 mm/sec, and maintained until a surface temperature of the lithium secondary battery reached about 40° C. or less. A degree of battery safety of each battery was represented as one of L1 to L5 according to the following criteria.

TABLE 1

| | Safety Evaluation | | |
|---|---|---|---|
| Example | 1^{st} penetration | 2^{nd} penetration | 3^{rd} penetration |
| Example 1 | L1 | L1 | L4 |
| Example 2 | L1 | L1 | L1 |
| Example 3 | L1 | L1 | L1 |
| Example 4 | L1 | L1 | L1 |
| Example 5 | L1 | L1 | L1 |
| Example 6 | L1 | L1 | L1 |
| Example 7 | L1 | L1 | L1 |
| Example 8 | L1 | L1 | L1 |
| Comparative | L4 | L4 | L4 |
| Example 1 | | | |
| Comparative | L4 | L4 | L4 |
| Example 2 | | | |
| Comparative | L4 | L4 | L4 |
| Example 3 | | | |
| Comparative | L4 | L4 | L4 |
| Example 4 | | | |
| Comparative | L4 | L4 | L4 |
| Example 5 | | | |

### —Battery Safety Evaluation Criteria

L1: leakage, L2: generation of heat of less than 200° C., L3: generation of heat of 200° C. or greater, L4: fire, L5: bursting

Referring to Table 1, the lithium secondary batteries of Examples 1 to 8 had improved safety, compared to the lithium secondary batteries of Comparative Examples 1 to 5, indicating that exothermic reaction was reduced in the lithium secondary batteries including the electrolytes to which the dithiocarbonyl compounds represented by Formula 1 were added, e.g., due to formation of a thin film with good stability on an electrode surface.

### Evaluation Example 2

### Evaluation of Charge-Discharge Characteristics

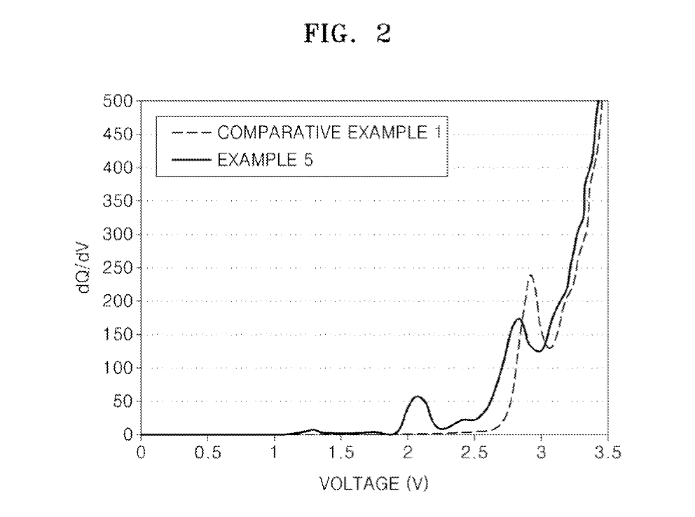

The lithium secondary batteries of Example 5 and Comparative Example 1 were each charged at a constant current of 0.2 C rate at about 25° C. to a voltage of about 4.2 V, and then cut off at a current of 0.05 C rate maintaining the constant voltage mode of about 4.2 V, and discharged at a constant current of 0.2 C rate to a voltage of about 2.8 V to evaluate initial charge-discharge characteristics of the lithium secondary batteries. Differential capacity (dQ/dv) curves at the first cycle of the lithium secondary batteries of Example 5 and Comparative Example 1 are shown in FIG. 2.

Referring to FIG. 2, the lithium secondary battery of Comparative Example 1 exhibited a reduction peak of the nonaqueous organic solvent at about 3 V.

The lithium secondary battery of Example 5 exhibited reduction peaks of 1-(methyldithiocarbonyl) imidazole at about 1.26 V and about 2.1 V, and a reduction peak of the nonaqueous organic solvent at about 2.8 V. The results may be attributed to the use of 1-(methyldithiocarbonyl) imidazole as an additive in the lithium secondary battery of Example 5, which reduced at a lower voltage than the nonaqueous organic solvent to form a modified SEI layer, and consequently suppressed reduction of the nonaqueous organic solvent.

### Evaluation Example 3

### Impedance Measurement

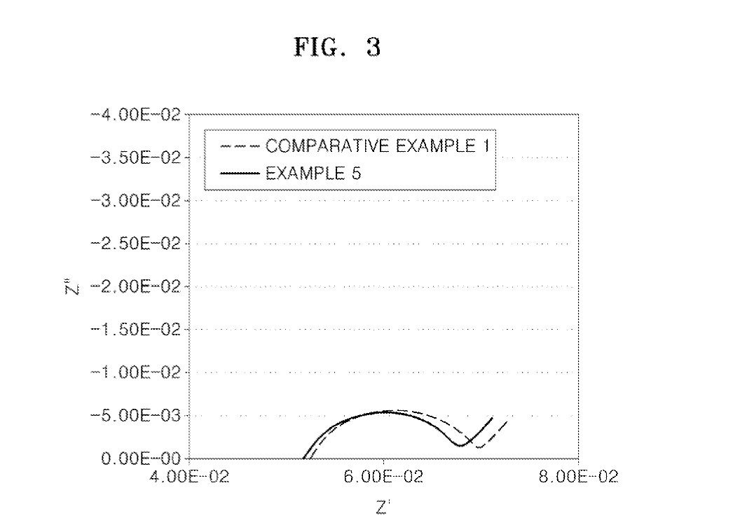

Impedances of the lithium secondary batteries of Example 5 and Comparative Example 1 were measured using an impedance analyzer (PARSTAT 2273) according to a 2-probe method. A frequency range for the impedance measurement was from about 10⁵ to about 10⁻¹ Hz. Nyquist plots obtained through the impedance measurement are shown in FIG. 3.

Referring to FIG. 3, the lithium secondary battery of Example 5 was found to have a lower impedance than that of the lithium secondary battery of Comparative Example 1. This may be attributed to the addition of the dithiocarbonyl-based compound in the lithium secondary battery of Example 5 causing the formation of the SEI layer with improved ion conductivity on an electrode surface, leading to equilibrium of internal materials.

### Evaluation Example 4

### Battery Safety Evaluation—Thermal Runaway Reaction Experiment

(Preparation of Electrolyte)

### Preparation Example 9

After LiPF₆ was dissolved to a concentration of 0.9 M in a mixed nonaqueous organic solvent of EC, EMC, and DMC in a volume ratio of 30:50:20, 6 parts by weight of FEC, 0.5 parts by weight of VEC, 2.5 parts by weight of SN, 0.2 parts by weight of LiBF₄, 3 parts by weight of PS, and 1 part by weight of 1-(methyldithiocarbonyl) imidazole (represented by Formula 10 above) were added as additives to prepare an electrolyte.

### Comparative Preparation Example 6

An electrolyte was prepared in substantially the same manner as in Preparation Example 9, except that 1-(methyldithiocarbonyl) imidazole (represented by Formula 10) was not added.

(Manufacture of Test Cells)

### Example 9

A positive electrode, a negative electrode, and a lithium secondary battery were manufactured in substantially the same manner as in Example 1, except that LiCoO₂ as a positive active material and the electrolyte of Preparation Example 9 were used.

### Comparative Example 6

A positive electrode, a negative electrode, and a lithium secondary battery were manufactured in substantially the same manner as in Example 9, except that the electrolyte of Comparative Preparation Example 6 was used.

(Thermal Runaway Reaction Experiment)

After each of the lithium secondary batteries of Example 9 and Comparative Example 6 (18650 cylindrical type, 2200 mAh) was charged to about 4.2 V at a state-of-charge of 100%, temperature changes of the cells were monitored using an accelerating rate calorimeter (ARC) while heating in an insulated state. ARC evaluation conditions are shown in Table 2, below.

TABLE 2

| Parameter setting | Set value | |
|---|---|---|
| Start Temperature | 25° | C. |
| End Temperature | 350° | C. |
| Slope Sensitivity | 0.05° | C./min |
| Heat Step Temperature | 5° | C. |
| Wait Time for Stabilization after Heating | 20 | min |

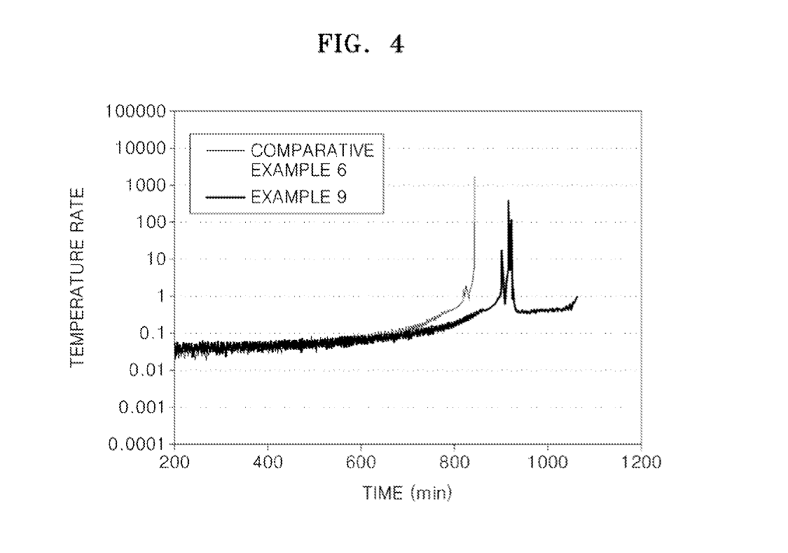

FIG. 4 illustrates a graph of temperature rate against time in the lithium secondary batteries (18650 standard cells) of Example 9 and Comparative Example 6, obtained using the ARC. The lithium secondary battery of Example 9 using the electrolyte including 1-(methyldithiocarbonyl) imidazole had a lower self-heat generation rate and took longer until thermal runaway occurred, compared to the lithium secondary battery of Comparative Example 6, which did not include 1-(methyldithiocarbonyl) imidazole. The amount of heat generation in the lithium secondary battery of Example 9 was smaller than that in the lithium secondary battery of Comparative Example 6, indicating a temperature rise time of the lithium secondary battery of Example 9 was delayed relative to that of the lithium secondary battery of Comparative Example 6. This supports that the addition of 1-(methyldithiocarbonyl) imidazole in the electrolyte of the lithium secondary battery may cause the formation of the SEI layer with improved high-temperature stability to suppress the occurrence of thermal runaway.

By way of summation and review, the nonaqueous organic electrolyte may be decomposed through reaction with a negative or positive electrode during charging and discharging, or may be spontaneously thermally decomposable. Such decomposition may cause heat generation in a battery and/or an explosion of the battery, which may occur when the amount of heat release is smaller than the amount of heat generation, and may occur more frequently in larger batteries.

An electrolyte additive for a lithium battery according to an embodiment may implement high capacity and may help improve the safety of the lithium battery.

The embodiments may provide an electrolyte additive for a lithium battery that may improve battery safety.

As described above, according to the embodiments, a lithium battery may include an electrolyte including a dithiocarbonyl-based compound as an additive. Consequently, the lithium battery may have improved safety due to suppressed exothermic reaction therein.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. An electrolyte additive for a lithium battery, the electrolyte additive comprising a dithiocarbonyl-based compound represented by Formula 1: 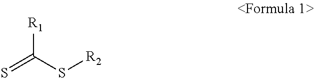
wherein, in Formula 1, R₁ comprises a substituted or unsubstituted alicyclic or aromatic hydrocarbon group, and R₂ comprises a substituted or unsubstituted aliphatic, alicyclic, or aromatic hydrocarbon group.

2. The electrolyte additive as claimed in claim 1, wherein:
the aliphatic hydrocarbon group is a C1-C20 alkyl group, a C2-C20 alkenyl group, or a C2-C20 alkynyl group,
the alicyclic hydrocarbon group is a C3-C20 cycloalkyl group, a C3-C20 cycloalkenyl group, or a C2-C20 heterocycloalkyl group, and
the aromatic hydrocarbon group is a C6-C20 aryl group or a C2-C20 heteroaryl group.

3. The electrolyte additive as claimed in claim 2, wherein
the C2-C20 heterocycloalkyl group and the C2-C20 heteroaryl group each independently comprise 1 to 3 heteroatoms.

4. The electrolyte additive as claimed in claim 3, wherein the heteroatoms comprise at least one of N, S, O, or P.

5. The electrolyte additive as claimed in claim 2, wherein:
the C2-C20 heterocycloalkyl group is a pyrolidinyl group, a piperidinyl group, a piperazinyl group, or a morpholinyl group,
the C6-C20 aryl group is a phenyl group or a naphthyl group, and
the C2-C20 heteroaryl group is a pyrrolyl group, an imidazolyl group, a pyrazolyl group, a benzimidazolyl group, a triazolyl group, a thiophenyl group, an indolyl group, a pyridinyl group, a pyridazinyl group, a pyrimidinyl group, or a quinolinyl group.

6. The electrolyte additive as claimed in claim 1, wherein R₁ is a pyrolidinyl group, a piperidinyl group, a piperazinyl group, a morpholinyl group, a phenyl group, a naphthyl group, a pyrrolyl group, an imidazolyl group, a pyrazolyl group, a benzimidazolyl group, a triazolyl group, or a thiophenyl group.

7. The electrolyte additive as claimed in of claim 1, wherein R₂ is a methyl group, an ethyl group, a propyl group, an isopropyl group, a butyl group, an isobutyl group, a tert-butyl group, a sec-butyl group, a vinyl group, an allyl group, a butenyl group, a pentenyl group, a phenyl group, or a benzyl group.

8. The electrolyte additive as claimed in claim 1, wherein the dithiocarbonyl-based compound represented by Formula 1 comprises at least one of compounds represented by Formula 2 to Formula 16:

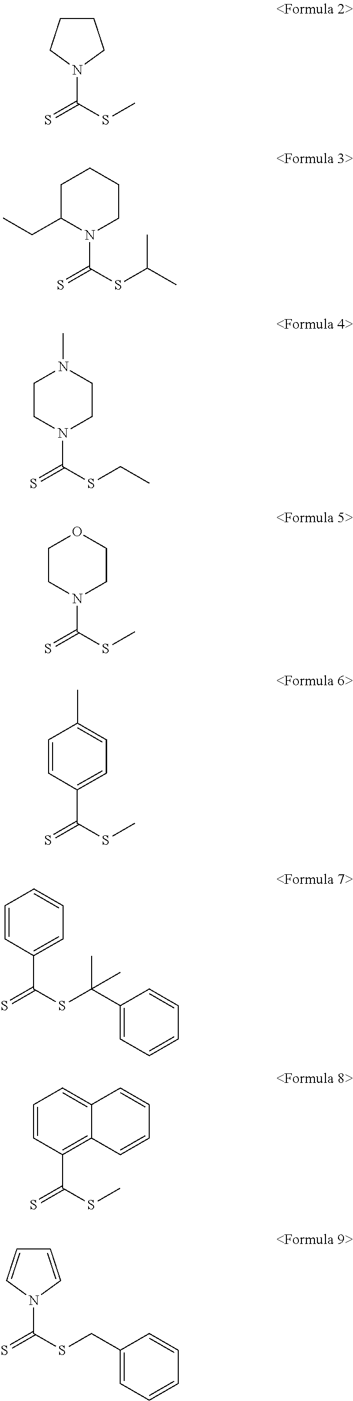

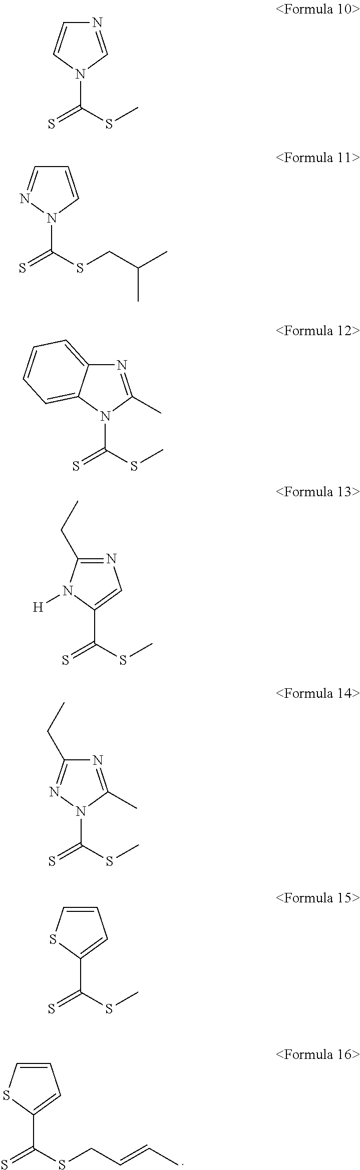

9. An electrolyte for a lithium battery, the electrolyte comprising:
a lithium salt;
a nonaqueous organic solvent; and
the electrolyte additive as claimed in claim 1.

10. The electrolyte as claimed in claim 9, wherein the electrolyte additive is included in the electrolyte in an amount of about 0.01 parts to about 5 parts by weight, based on 100 parts by weight of the nonaqueous organic solvent.

11. The electrolyte as claimed in claim 10, wherein the electrolyte additive is included in the electrolyte in an amount of about 0.1 parts to about 2 parts by weight, based on 100 parts by weight of the nonaqueous organic solvent.

12. The electrolyte as claimed in claim 9, wherein the lithium salt comprises at least one of LiPF₆, LiBF₄, LiAsF₆, LiClO₄, LiCF₃SO₃, LiSbF₆, CF₃SO₃Li, LiN(SO₂CF₃)₂, LiC₄F₃SO₃, LiAlF₄, LiAlCl₄, LiN(SO₂C₂F₅)₂, LiN(CₓF₂ₓ₊₁SO₂)(C_{y}F_{2+y}SO₂), in which x and y are natural numbers, LiCl, and LiI.

13. The electrolyte as claimed in claim 9, wherein the nonaqueous organic solvent comprises a carbonate-based compound, an ester-based compound, an ether-based compound, a ketone-based compound, an alcohol-based compound, an aprotic compound, or a combination thereof.

14. The electrolyte as claimed in claim 13, wherein the nonaqueous organic solvent comprises the carbonate-based compound, the carbonate-based compound including a chain carbonate compound, a cyclic carbonate compound, a chain fluorocarbonate compound, a cyclic fluorocarbonate compound, or a combination thereof.

15. The electrolyte as claimed in claim 9, wherein the nonaqueous organic solvent comprises a chain carbonate compound and a cyclic carbonate compound.

16. The electrolyte as claimed in claim 15, wherein the cyclic carbonate is included in the electrolyte in an amount of at least about 5 vol. %, based on a total volume of the nonaqueous organic solvent.

17. The electrolyte as claimed in claim 9, further comprising at least one additive selected from the group of tris(trimethylsilyl) phosphate (TMSPa), lithium difluorooxalate borate (LiFOB), vinylene carbonate (VC), fluoroethylene carbonate (FEC), vinyl ethylene carbonate (VEC), propane sultone (PS), succinonitrile (SN), LiBF₄, a silane compound having a functional group that forms a siloxane bond, and a silazane compound.

18. The electrolyte as claimed in claim 9, further comprising propane sultone (PS), succinonitrile (SN), LiBF₄, or a combination thereof.

19. A lithium battery, comprising:
a positive electrode;
a negative electrode; and
the electrolyte as claimed in claim 9.
